(19) United States
(12) Patent Application Publication
(10) Pub. No.: US 2011/0017839 A1
(43) Pub. Date: Jan. 27, 2011

(54) Fragrance diffusion device

(76) Inventor: Samantha S. Biscardi, Wayne, NJ (US)

Correspondence Address: RICHARD R. MUCCINO, 758 SPRINGFIELD AVENUE, SUMMIT, NJ, 07901, US

(21) Appl. No.: 12/460,514

(22) Filed: Jul. 21, 2009

(51) Int. Cl. A61L 9/02

(52) U.S. Cl. 239/44

(57) ABSTRACT

The present invention provides devices for dispensing a volatile substance into an atmosphere employing a novel heat source to promote the release of the volatile substance. The present invention also provides methods for dispensing a volatile substance into an atmosphere employing the novel device.

### FIELD OF THE INVENTION

The present invention provides devices for dispensing a volatile substance into an atmosphere employing a novel heat source to promote the release of the volatile substance. The present invention also provides methods for dispensing a volatile substance into an atmosphere employing the novel device.

### BACKGROUND OF THE INVENTION

The prior art discloses fragrance diffusion devices that usually fall within one of the following categories. Burnable devices include candles, wax pots, and potpourri pots. Essentially, these devices include any product that utilizes an open flame as a method of generating fragrance diffusion through heat. Plug in devices include products that use electrical power to generate heat and diffuse the fragrance or use a drive technology to deliver the fragrance. Examples include plug in heated units, plug in membrane driven units, and fan driven units, which circulate air over a fragranced wick system. Non-powered diffusers include devices that take the form of a fragrance oil in a container, with bamboo or similar reeds that absorb the fragrance and diffuse it under ambient conditions. These devices include paperboard air fresheners such as those used in automobiles or fragranced gels. In these devices, one of the major limitations is performance due to the lack of volatility and air movement in the vapor phase of the fragrance.

U.S. Pat. No. 7,377,493 (Thomas) discloses a system for emanating fragrance vapor into a region. A fragrance liquid within a receptacle exhibits a liquid phase-vapor phase interface surface when in a quiescent state. Utilizing sub-pressure generators such as a fan driven system, the pressure above the interface is lowered to promote the escape of vapor molecules for dissemination into the atmosphere. The surface area of the fragrance fluid may be increased through the use of a bubbling technique to derive an effective area permitting a larger vapor molecule escape.

U.S. Pat. No. 7,303,143 (Davis et al.) discloses a wick assembly provided for use in a dispensing device that dispenses volatile material from a wick by heat, plug in ring heater. Two wick sections of differing material are provided in stacked fashion with a coupler sleeve that can be used to facilitate attachment of the wick structure to a reservoir. One wick section preferably provides control over the rate of dispensing and the other provides improved thermal resistance and resistance to clogging.

U.S. Pat. No. 6.830,733 (Stanley, III) discloses an artificial flower, which includes a hollow stem and a flower portion disposed adjacent to one end of the hollow stern. A fragrance source is associated with the artificial flower. In one embodiment, the fragrance source is disposed within the hollow stem of the artificial flower and the fragrance source is adapted to emit a fragrance that moves through at least a portion of the hollow stem.

U.S. Pat. No. 6,249,645 (Smith) discloses a scent device that is adapted for use in a vehicle. The device includes a bowl having a heater element adapted for use in a vehicle, the bowl being suitable for use with various types of scent supplies that are not otherwise usable in vehicle air fresheners. A potpourri container is included for vehicle operation, that has a heater element, and a bowl, thermally coupled and configured to a cigarette lighter, including a cavity designed to hold a plurality of scents, and a quantity of solution. A cover is positioned over the bowl to seal the cavity, having a sunken section, located centrally to the cover. Pores, located through the cover, allow vapor to escape from the cavity. A lip is positioned on a bottom side of the cover, designed to fit within the bowl and hold the bowl in place. A handle is used for easy removal of the bowl.

U.S. Pat. No. 5,725,152 (Akyu) discloses an air freshener dispenser provided with an emanator medium, which is easily replaced, allowing for sustainable use of the container. The dispenser has a head section consisting of a head assembly and a fixing member. The head assembly is detachably connected to the container and comprises a cylindrical cap member having a plurality of threads for mating with threads on the outer periphery of the container neck and an exterior ornamental part extending upwardly and outspreadly from the outermost periphery of the cap member. The fixing member is inserted into the space surrounded by the thread in such a way that the emanator medium is firmly held there between, its base being in tight contact with the wick, which carries the fragrant liquid by capillary action.

U.S. Pat. No. 5,651,942 (Christensen) discloses a device for supplying aromatic fragrances to the atmosphere employing a heating element, plug in unit, in a container to heat a fragrance base material contained in a receptacle to supply a pleasant aroma to the atmosphere, primarily to eliminate bathroom and kitchen odors.

U.S. Pat. No. 5.437.410 (Babasade) discloses a passive aromatic substance dispenser. A highly absorbent rod shaped wick, held within a hermetically sealed tube, serves as a fragrance dispensing reservoir in contact with an efficient fragrance diffuser, and also serves as a means for suspending the diffuser in a selected area to be fragranced. When fragrancing an area is not desired, closing the housing for the diffuser seals the diffuser from ambient air currents, protecting the aromatic substance from evaporating from the diffuser. The vacuum that develops within the tube due to the absorption of aromatic substances from the rod and into the diffuser prevents draining of the fragrance material from the rod and into the diffuser when not required, thus providing self regulation of the fragrance material by the dispenser.

U.S. Pat. No. 5,175,791 (Muderlak et al.) discloses a fragrance diffuser comprising a housing defining a chamber for holding a fragrance-emitting block, a heating element located within the housing, a timing and indicating circuit board to signal when the predicted life of the fragrance emitting block has expired, a power supply connection plug for providing electrical power to the heating element and the timing and indicating board, and a keying system incorporated in the chamber and on the fragrance-emitting blocks. The blocks are encoded or keyed to indicate that they possess particular predefined characteristics, such that only blocks having those characteristics may be inserted. The diffuser is constructed with a downwardly projecting housing extension for the power supply connection to allow the use of an adjacent electric power outlet when the diffuser is properly installed. The diffuser also includes an indicator LED to indicate when a timing interval, corresponding to the life of a fragrance block inserted therein, has expired, wherein the timing interval is reset by a switch only at the request of a user. Various circuit embodiments are disclosed for increasing the power applied to the heating element over time since the aroma strength of a fragrance block decreases over time.

United States Design patent no. 384,143 (Racinelli) discloses 6 Figures for ornamental designs for a potpourri simmer vase.

United States Design patent no. 13,619 (Tweeddalei) discloses 1 Figure for an ornamental design for a vase.

United States Patent Application no. 2008/016922 (Gaines) discloses a packaged kit for storing a fragrance dispensing assembly that includes a storage container having a sealed interior chamber containing porous reeds in a fragranced liquid. The porous reeds and the fragranced liquid are put in the storage container, placed in contact with each other, and the storage container is sealed. The packaged kit provides instant and continuous fragrance to a room by opening the sealed storage container and transferring the porous reeds and the fragranced liquid from the storage container to a holding container. A portion of the porous reeds extends from the holding container and releases the fragrance from the exposed fragrant liquid into the surrounding area.

United States Patent Application no. 2007/0257128 (Brown et al.) discloses an apparatus adapted to disseminate volatile liquid into an atmosphere. The apparatus comprises an evaporation element having an evaporation surface bearing a capillary channel and a wick. The wick makes contact with the evaporation element at a surface other than the one bearing the capillary channel. Liquid transfer contact between wick and capillary channel is provided by means of a capillary passage passing from that part of the evaporation element contacting the wick to the capillary channel. The evaporation element is preferably a capillary sheet. The apparatus allows the use of wicks that are smaller than usual, with no loss in efficiency of dissemination.

United States Patent Application no. 2006/0193611 (Ruiz Ballesteros et al.) discloses a vaporizer device which is connected to an electric mains by means of a plug, and incorporates a single container equipped with a body of two or more independent receptacles, which hold different liquid fragrances, which respective wicks are immersed and a lid which closes the body and renders the receptacles independent. The lid has openings in correspondence with the positions of the wicks for the egress and vaporization of the fragrance by actuation of one or more resistance elements located in opposition to the openings by means of a manual pushbutton or automatically in accordance with a pre-established program during the fragrances in a sequential or combined manner during predetermined intervals of time.

WO 2007045834 (Goreham et al.) discloses a device for dispensing a fluid for fragrancing, deodorizing, or sanitizing air. The device has a body comprising a shade, a chassis having a base, a cartridge housing mounted on the chassis and adapted for housing, a cartridge containing a fluid to be dispensed, and a plug in electrical light source. The electrical light source is located on the base between the cartridge housing and a sidewall of the body.

JP2091204 (Kenji) discloses a flower arrangement stand to dispense the smell of potpourri by placing the potpourri in a vessel and providing the vessel with an aeration means for sending the smell of the potpourri and another means for exchanging the potpourri. A pipe is extended upward from the bottom of the clear potpourri vessel. The top height of the pipe is made about 3 cm higher than the top of the potpourri vessel. When a potpourri is placed in the pipe, a space is formed between the top of the vessel and the pipe and the space serves as a conduit for the potpourri smell. When the smell of the potpourri is exhausted, the potpourri is taken out of the pipe and the vessel is turned upside down whereby the potpourri can be readily taken out. Fresh potpourri can be readily placed into the vessel.

Therefore, there is a need to develop improved devices for dispensing a volatile substance into an atmosphere employing a heat source to promote the release of the volatile substance.

### IN THE FIGURES

FIG. 1 depicts one embodiment of the device for dispensing a volatile substance into an atmosphere employing a battery power device.

FIG. 2 depicts another embodiment of the device for dispensing a volatile substance into an atmosphere employing a solar power device.

### SUMMARY OF THE INVENTION

The present invention provides a device for dispensing a volatile substance into an atmosphere, wherein the device comprises: (a) a reservoir containing a volatile substance; (b) at least one wicking device in contact with the volatile substance extending from the reservoir; (c) a container in which the reservoir is mounted; and (d) a heating device thermally coupled to the container capable of heating the volatile substance in the reservoir.

The present invention further provides a method for dispensing a volatile substance into an atmosphere, wherein the method comprises the sequential steps of: (1) providing a device for dispensing a volatile substance into an atmosphere, wherein the device comprises: (a) a reservoir containing a volatile substance; (b) at least one wicking device in contact with the volatile substance extending from the reservoir; (c) a container in which the reservoir is mounted; and (d) a heating device thermally coupled to the container capable of heating the volatile substance in the reservoir; and (2) activating the heating device to heat the volatile substance in the reservoir to thereby dispense the volatile substance into the atmosphere.

### DETAILED DESCRIPTION OF THE INVENTION

The present invention provides novel fragrance diffusion devices. The fragrance diffusion devices improve the fragrance delivery performance of wicking devices by using heat as part of the delivery system. The fragrance diffusion devices may be in the form of a reservoir, such as a vase, containing a volatile substance and a wicking device, such as one or more absorbent reeds, in contact with the volatile substance extending from the reservoir. The reservoir is mounted in a container and a heating device, such as a battery power device or a solar device, which is coupled to the container and is capable of heating the volatile substance in the reservoir. An important advantage of a battery power device or a solar power device over a plug in device is safety and portability. The present invention also provides a method for dispensing a volatile substance into an atmosphere. The method comprises providing the novel device of the invention for dispensing a volatile substance into an atmosphere and activating the heating device to heat the volatile substance in the reservoir to thereby dispense the volatile substance into the atmosphere. The temperature of the warmed volatile substance would be low enough to be safe (<about 115° F.), but warm enough to enhance performance and delivery of the volatile substance.

The invention will be better understood from the following detailed description of the preferred embodiments taken in conjunction with the Figures, in which like elements are represented by like referenced numerals.

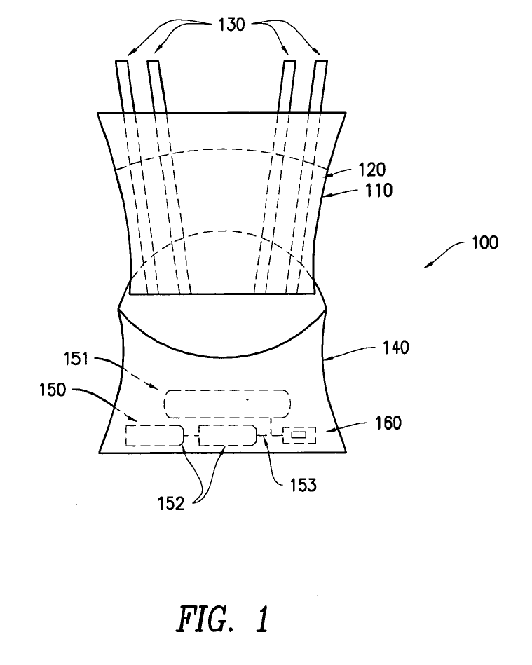

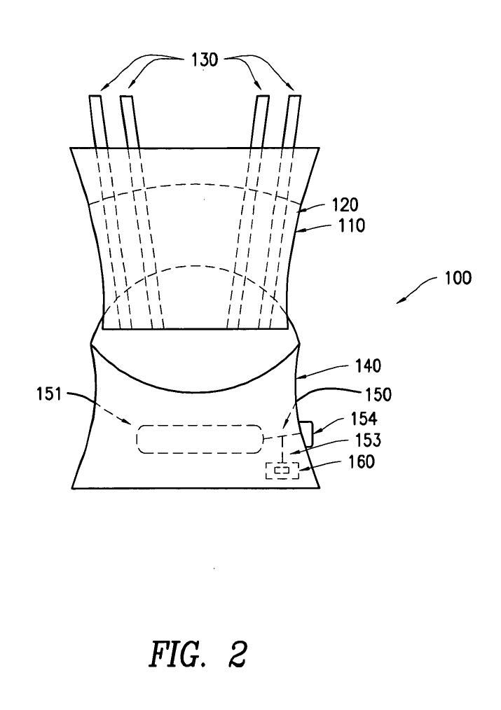

In FIGS. 1 and 2, device (100) for dispensing a volatile substance into an atmosphere comprises a reservoir (110) containing a volatile substance (120). At least one wicking device (130) is in contact with the volatile substance (120) extending from the reservoir (110). In FIGS. 1 and 2, wicking device (130) is shown as absorbent reeds. Reservoir (110) is mounted in container (140). In FIG. 1, heating device (150) is thermally coupled to container (140) capable of heating the volatile substance in the reservoir. In FIG. 1, heating device (150) is a warming plate (151) and a battery power device (152) connected with thermal couple (153). In FIG. 2, heating device (150) is a warming plate (151) and a solar power device (154) connected with thermal couple (153). Reservoir (110) and container (140) may be of any type, shape or size, to suit the aesthetics of device (100). Optionally, the heating device may further comprise switch (160) to turn the heating device on or off. Switch (160) can be a manual switch or a photosensor switch.

The reservoir is a hollow vessel, which contains the volatile substance and can be of any desired shape. The volatile substance is a substance that has a tendency to readily vaporize. At a given temperature, substances with higher vapor pressures will vaporize more readily than substances with lower vapor pressures. Although the term is usually applied to liquids, volatility can apply to solid materials also. A preferred volatile substance is a fragrance agent, also known as an odorant or an aroma. Fragrance agents can be found in food, wine, spices, perfumes, fragrance oils, and essential oils.

The term “wicking device” refers to any absorbent material that is capable of acting as a wick, that is, of causing the volatile substance in the reservoir to be transported from the reservoir along the wick to a location where evaporation will take place. The wick may be, for example, one of the cylindrical types well known to the art, or it may be a piece of flat absorbent material, provided the wick performs its required function, there is no limit as to material or construction. Typical materials include porous plastics, porous ceramics, compacted fibrous materials, cardboard, and the like. The skilled person can select a suitable material for any desired end-use. There may be present more than one wick. In addition, the wick can be any desired shape. While the traditional cylindrical or frustaconical wicks well known to the art are especially useful because of their ready availability and cheapness, any other practical or decorative shape may be used. The wicking device may also be a reed, preferably made of wood rattan or bamboo. Reeds are particularly desirable since they have aesthetic qualities and large surface areas. The container, like the reservoir is a hollow vessel, in which the reservoir is mounted and can be heated.

The heating device thermally coupled to the container capable of heating the volatile substance in the reservoir is preferably a battery power device or a solar power device, and more preferably is a battery power device. An important advantage of a battery power device or a solar power device over a plug in device is safety and portability. The heating device thermally coupled to the container capable of heating the volatile substance in the reservoir heats the volatile substance to a temperature about <115° F.

A battery power device, or voltaic cell, is a combination of one or more electrochemical Galvanic cells, which store chemical energy that can be converted into electric potential energy, creating electricity. Batteries are classified into two broad categories, each type with advantages and disadvantages. Primary batteries irreversibly (within limits of practicality) transform chemical energy to electrical energy. When the initial supply of reactants is exhausted energy cannot be readily restored to the battery by electrical means. Secondary batteries can be recharged, that is, they can have their chemical reactions reversed by supplying electrical energy to the cell, restoring their original composition. One skilled in the art can choose the appropriate type of battery power device.

Solar energy is the radiant light and heat from the Sun or indoor lighting that uses a range of ever-evolving technologies. Solar technologies are broadly characterized as either passive solar or active solar depending on the way they capture, convert and distribute light. Active solar techniques include the use of photovoltaic panels, solar thermal collectors, with electrical or mechanical equipment, to convert sunlight into useful outputs. Passive solar techniques include orienting an object to the light, selecting materials with favorable thermal mass or light dispersing properties, and designing spaces that naturally circulate air. The device of the invention for dispensing a volatile substance into an atmosphere could be placed next to a window, for example. One skilled in the art can choose the appropriate type of solar power device.

The heating device may further comprise a switch to turn the heating device on or off. The switch can be a manual switch or a photosensor switch. A manual switch is an electrical component, which can break an electrical circuit. The most familiar form of switch is a manually operated electromechanical device with one or more sets of electrical contacts. A photosensor switch is a device that measures a physical quantity of light and converts it into a signal, which can be read by an instrument to break an electrical circuit.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

1. A device for dispensing a volatile substance into an atmosphere, wherein the device comprises:
(a) a reservoir containing a volatile substance;
(b) at least one wicking device in contact with the volatile substance extending from the reservoir;
(c) a container in which the reservoir is mounted; and
(d) a heating device thermally coupled to the container capable of heating the volatile substance in the reservoir.

2. The device according to claim 1, wherein the wicking device in (b) is a reed.

3. The device according to claim 1, wherein the heating device in (d) is a battery power device or a solar power device.

4. The device according to claim 3, wherein the heating device is a battery power device.

5. The device according to claim 1, wherein the heating device thermally coupled to the container capable of heating the volatile substance in the reservoir heats the volatile substance to a temperature about <115° F.

6. The device according to claim 1, wherein the heating device further comprises a switch to turn the heating device on or off, wherein the switch can be a manual switch or a photosensor switch.

7. A method for dispensing a volatile substance into an atmosphere, wherein the method comprises the sequential steps of:
(1) providing a device for dispensing a volatile substance into an atmosphere, wherein the device comprises:
(a) a reservoir containing a volatile substance;
(b) at least one wicking device in contact with the volatile substance extending from the reservoir;
(c) a container in which the reservoir is mounted; and
(d) a heating device thermally coupled to the container capable of heating the volatile substance in the reservoir; and
(2) activating the heating device to heat the volatile substance in the reservoir to thereby dispense the volatile substance into the atmosphere.

8. The method according to claim 7, wherein the wicking device in (b) is a reed.

9. The method according to claim 7, wherein the heating device in (d) is a battery power device or a solar power device.

10. The method according to claim 9, wherein the heating device is a battery power device.

11. The method according to claim 7, wherein the heating device thermally coupled to the container capable of heating the volatile substance in the reservoir heats the volatile substance to a temperature about <115° F.

12. The method according to claim 7, wherein the heating device further comprises a switch to turn the heating device on or off, wherein the switch can be a manual switch or a photosensor switch.
